(19) United States
(12) Patent Application Publication
(10) Pub. No.: US 2026/0248416 A1
(43) Pub. Date: Aug. 27, 2026

(54) VIDEO ANALYSIS GAIT ASSESSMENT SYSTEM AND METHOD

(71) Applicant: DUBLIN CITY UNIVERSITY, Dublin (IE)

(72) Inventor: Aidan BORAN, Blossom Hill (IE)

(21) Appl. No.: 18/877,302

(22) PCT Filed: Jun. 29, 2023
(86) PCT No.: PCT/EP2023/067934

§ 371 Date: Dec. 20, 2024

(30) Foreign Application Priority Data
Jul. 1, 2022 (GB) 2209727.3

(51) Int. Cl. A61B 5/11; A61B 5/00; G06V 40/20

(52) U.S. Cl. CPC A61B 5/112; A61B 5/7435; G06V 40/25

(57) ABSTRACT

A computer implement method of gait analysis comprising carrying out video analysis using augmented or virtual reality markers without the use of external sensors or markers which allows for objective longitudinal comparison of gait analyses and provides a flexible system allowing multiple gait analysis test to be carried out using only a single test system.

### CROSS-REFERENCE TO RELATED APPLICATIONS

The present application is a U.S. National Phase patent application of International Patent Application No. PCT/EP2023/067934, filed on Jun. 29, 2023, which claims the benefit of Great Britain application no. GB2209727.3 filed on Jul. 1, 2022, the benefit of which is claimed and the disclosures of which are incorporated herein by reference in their entirety.

### FIELD OF THE DISCLOSURE

The present disclosure relates to a video analysis gait assessment system and method. More particularly, but not exclusively, it relates to a video analysis gait assessment system and method requiring no markers to be placed upon a subject or surrounding environment such as floors, walls or ceilings.

### BACKGROUND OF THE DISCLOSURE

Gait analysis is a useful tool in screening for identifying biomarkers associated with a number of diseases and debilitating conditions. As the world's population ages there is an increasing percentage of the population that suffer from frailty, defined as the group of older people who are at highest risk of adverse outcomes such as falls, disability, admission to hospital, or the need for long-term care. Currently, in Ireland over 40% of the healthcare budget is directed to dealing with conditions in those over 65 years of age.

The analysis of a person's gait is typically carried out either by a visual assessment by a trained professional or by a technology based assessment. Visual assessments are inherently subjective whatever the level of training of the professional. Such visual gait assessment are therefore be unreliable when considering consistency between professional assessments over time and when different professionals are carrying out the assessments.

In view of this a number of technological solutions have been created for carrying out gait analysis for example the Gait Rite™ system uses a pressure mat to measure a user's footfall to determine issues with mobility and balance etc. Such a system is unwieldy, typically being stored in a container the size of a large suitcase, and must be deployed correctly for each use. The issue with deployment typically leads to systems remaining in situ for an extended period and requiring that the user is brought to the system which can be problematic for older, frail or infirm users. Other systems such as Kinesis™ use Bluetooth enabled sensors strapped to the user in order to measure the motion of points upon the user's body as they walk to provide data for gait analysis. Such Bluetooth sensor based systems typically sample at between 2 k and 3 k signals per second which results in large amount of data above what is required for the gait analysis leading to data storage issues and issues around noise removal. An alternative is to have wired sensors strapped to the user which is cumbersome, can alter the user's gait and is potentially dangerous for an elderly or infirm user as they can represent a trip hazard. All of these sensor based systems are complex and computationally intensive.

US 2014/0276130 discloses a virtual reality (VR) based gate analysis system with sensors attached to patients. VR based sensing arrangements have safety issues due to the headsets required and older persons tolerate them particularly badly due to age related changes in their inner ears requiring visual stimuli for balance. US 2021/059565 discloses gait-based assessment of neurodegeneration, which requires a machine learning model to be trained for each specific disease to be assessed and as such is not scalable. Furthermore, it uses a single camera without a synthesis of depth from other elements and therefore only a two dimensional generation and analysis of keypoint, joints, is possible. US 2021/0357021 discloses an AR system for gait therapy rehabilitation therapy in which Bluetooth enabled sensors are mounted upon a user's feet that monitor the user's movement relative to generated augmented reality (AR) objects is measured. A VR like arrangement in which a smartphone is mounted in goggles is used to provide feedback to the user. WO 2020/018469 discloses a two-dimensional key-point analysis technique. WO 2019/210087 discloses a VR headset and sensor arrangement for testing visual functions of a user, this has the inherent safety and sensor data volume transmission issues noted in respect of other systems hereinbefore.

Video analysis of a user's gait is known but this requires either a static set up where reference points are physically provided against which the user's motion can be referenced and/or that the user wears a number of markers about their person, for example at joints and extremities, such that video capture has reference points from which to analyse the user's gait. The requirement for physical external reference points in current video gait analysis systems limits the broad applicability of these systems and requires the user to travel to a set location having predefined physical makers for the gait analysis, which can be problematic for elderly and infirm users.

A common feature of all gait analysis systems is that they are intractable in that developing new models that analyse the user's gait for a specific condition is complex and unwieldy due to the very complex programming required by them to identify a condition or syndrome. This leads to the need for multiple, complex and expensive systems having to be used to diagnose for a range of conditions. Dual task gait assessment types are not supported by any current gait assessment systems.

### SUMMARY OF THE DISCLOSURE

According to a first aspect of the present disclosure there is provided a gait analysis system comprising a video camera and a processor, the video camera and the processor being in communication and wherein the video camera is arranged to capture one or more video images of a subject's gait, and the processor being arranged to process a data structure corresponding to the video frames and the reference points to impart perspective to the video images at a, or the, processor, the processor being further to be configurable to define test parameters associated with video capture for a plurality of gait assessments.

The processor may be arranged to perform gait analysis based solely on the one or more video images. The system may be arranged to perform gait analysis where neither the subject nor the subject's environment comprises external physical markers denoting points to be monitored. The processor may be arranged to analyse the one or more video images to determine a condition of the subject.

The present disclosure provides a system that can be used without external markers or sensors in order to determine a condition of the subject. Thereby providing gate assessment as a service (GaaS) and that can provide customisable coaching instructions to the user about how to correctly capture the video for analysis

The ability to configure a wide range of existing/known gait assessment types means that an institution requires a single analysis system to diagnose a number of conditions.

The processor may be configurable to generate an augmented or virtual representation of test parameters upon a screen associated with the gait analysis system.

The use of augmented or virtual reality representations of test parameters allows removes the need for external markers, and specialist waymarked mats or pressure sensors with which to conduct gait analysis.

The system may comprise a data storage device linked to the processor, the data storage device being arranged to store a plurality of data sets each of which corresponds to an analytic technique. The system may comprise the user interface may be arranged to allow selection of one or more allow selection of any one or combination of and/or test parameters via the user interface. The processor may be arranged to run the one or more pre-configured computational analytic techniques on the captured video images. It will be appreciated that the analytic technique may comprise a diagnostic test arranged to diagnose, by way of non-limiting example, a disease and/or a condition and/or a syndrome. Alternatively, or additionally, the analytic technique may be arranged to, by way of non-limiting example, analyse a user's gait in respect of sporting performance and/or determine the identity of a user by comparison to a library of known users' gaits.

This allows a user to change or combine gait assessment types without the need for expert coding, thereby simplifying the use of the system.

The system may comprise a user input device comprising a user interface arranged to receive user inputs to define augmented or virtual reality objects to be used in the gait analysis. The user interface may further be arranged to define the placement of the augmented or virtual reality objects and/or their behaviour within a data structure comprising the one or more video images. The processor may be arranged to use the augmented or virtual reality objects as reference points from which to determine one or more key points location in three dimensions from the one or more video images. Movement of the key points may form the basis of the gait analysis configured computational analytic techniques.

The user interface may be arranged to allow selection of analysis at least one portion of the subject. The portion of the subject may comprise a set of the key points. The key points may be associated with one or more joints between bones of a subject. The portion may comprise a fraction of the subject. The fraction of the subject may comprise an upper portion of subject above a datum. The portion may comprise a fraction of the subject. The fraction of the subject may comprise an upper portion of subject below a datum.

The user interface may be arranged to allow selection of any one or combination of pre-configured computational analytic techniques to be run by the processor upon the captured video.

According to a second aspect of the present disclosure there is provided a processor arranged to act as the processor of the first aspect of the present disclosure.

According to a third aspect of the present disclosure there is provided a computer implement method of gait analysis of a subject comprising:
i) defining a gait analysis test via a user interface of a user device;
ii) generating augmented or virtual reality reference points associated with a test path at a processor based on the gait analysis test defined in step (i);
iii) recording a sequence of video frames of the subject perambulating;
iv) processing a data structure corresponding to the video frames and the reference points to impart perspective to the video images at a, or the, processor.

The processor may be configurable to define test parameters associated with video capture for a plurality of gait assessments.

The processor generate an augmented or virtual representation of test parameters upon a screen associated with the gait analysis system.

The method may comprise storing a plurality of data sets each of which corresponds to an analytic technique at a data storage device associated with the processor. The method may comprise allowing selection of any one or combination of and/or test parameters via the user interface. The method may comprise running the one or more pre-configured computational analytic techniques on the captured video images.

The method may comprise receiving user inputs to define augmented or virtual reality objects to be used in the gait analysis via the user interface. The method may further comprise defining the placement of the augmented or virtual reality objects and/or their behaviour within the data structure via the user interface. The method may comprise using the augmented or virtual reality objects as reference points from which to determine one or more key points location in three dimensions from the one or more video images.

The method may comprise selective of analysis of at least one portion of the subject. The portion of the subject may comprise a set of the key points. The key points may be associated with one or more joints between bones of a subject. The portion may comprise a fraction of the subject. The fraction of the subject may comprise an upper portion of the subject above a datum. The fraction of the subject may comprise an upper portion of the subject below a datum.

The method may comprise selecting any one or a combination of pre-configured computational analytic techniques stored on a data storage device associated with the processor to be run by the processor upon the captured video at step (i).

The method may comprise performing gait analysis based solely on the one or more video images. The method may comprise performing gait analysis where neither the subject nor the subject's environment comprises external physical markers denoting points to be monitored. The method may comprise analysing the video frames and/or data structure to determine a condition of the subject based upon the gait analysis the defined in step (i).

According to a fourth aspect of the present invention there is provided a non-transitory computer readable medium comprising instructions which, when executed, cause a processor to execute a method according to the third aspect of the present invention.

### BRIEF DESCRIPTION OF THE DRAWINGS

The present disclosure will now be described, by way of example only, with reference to the accompanying drawings, in which:

FIG. 1 is a schematic representation of a gait analysis system according to an aspect of the present disclosure;

FIG. 2 is schematic representation of augmented reality gait test layout produced by a processor according to an aspect of the present disclosure, showing the augmented reality assets and the event associated therewith;

FIG. 3 is a schematic representation of a mobile device running an application and a server of the system of FIG. 1;

FIG. 4 is a flow diagram showing the processing of video data of a gait analysis test according to an aspect of the present disclosure; and

FIGS. 5a and 5b show sub-sets of keypoint data of video data acquired using a camera of the system of FIG. 1.

### DETAILED DESCRIPTION OF DISCLOSURE

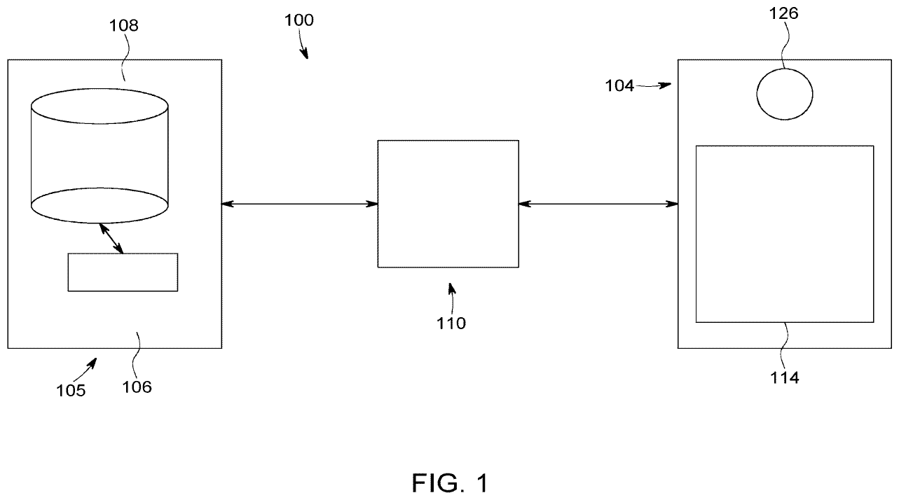

Referring now to FIG. 1, a gait analysis system 100 comprise a video camera 102, a user device 104, a server 105 comprising a processor 106 and a data storage device 108.

The camera 102 can, by way of non-limiting example, be a standalone video camera or it may form part of a mobile device such as a mobile phone.

The user device 104 may be a laptop computer, a mobile telephone or a tablet. The user device 104 may, but does not have to, house the camera 102.

The processor 106 is a typically a cloud based processor, for example a server in a data centre or it can be a locally based processor, for example on a PC or a laptop. The data storage device 108 is connected to the processor 106 either via a hardwired connection or via a wireless network connection.

In an embodiment where the camera 102 is separate from the user device 104, the camera 102, the user device 104 and the processor 106 are connected via a network 110. The network 110 may be a wireless or a wired network and many comprise the Internet.

In an embodiment where the camera 102 forms part of the user device 104, the user device 104 and the processor 106 are connected via a network 110. The network 110 may be a wireless or a wired network and many comprise the Internet. Typically, the camera 102 is a single, monocular camera.

The user device 104 allows the input of gait test configurations and gait parameter calculations via a user interface using a declarative language, for example “GaitKeeper Test Declarative Language” (GTDL). GTDL will be referenced as the declarative language of choice hereinafter, although the skilled person will appreciate that an equivalent language could be envisioned even though none is currently known. In one embodiment, JSON is used to represent the GTDL files, represents asset meshes using USDZ file format. GTDL files and associated assets are loaded from the server 105 using a RESTapi. Table 1 shows an exemplary GTDL language schema.

TABLE 1

| | |
|---|---|
| Test Name | The name of the test configuration. |
| | This is used to present the test name in the user interface. |
| | [String of four or less characters] |
| Test | A textual description of what this assessment does. |
| Description | It is used to show an information dialog in the user |
| | interface. |
| | [String: 0-200 characters] |
| AssetName | The name of the asset that the GaitKeeper system uses to |
| | identify, render and assign behaviours. It is also used to |
| | render the Icon name on the user interface |
| | [One of StartAsset, EndAsset or Waypoint] |
| Type | The name of the asset that the GaitKeeper system uses to |
| | identify, render and assign behaviours. |
| | [One of SA, EA, WP] |
| Cardinality | How many of this asset type are allowed in the system. |
| | GaitKeeper checks that only one Start and End are added. |
| | [Integer: 1] |
| Mandatory | A Boolean flag to check whether this asset is mandatory |
| | [Boolean: True or False] |
| Visibility | An enumerated value to indicate when the Asset becomes |
| | visible |
| | [String: One of ALL, START, END or WP] |
| Mesh | URL for the associated 3D object that represents this |
| | Asset. |
| | (The mesh type is limited to USDZ and OBJ types. It must |
| | contain all colour and material information) |
| Position | The real world position to place this asset, represented in |
| | metres from the position of the camera. |
| | [Point (x, y, z)] |
| Physics | Boolean value to indicate if this asset participate in |
| | occlusion and physics behaviours. |
| | [Boolean: True or False] |

GTDL allows the setup of both predefined and user defined test configuration files. Additionally, new test configuration files, written in GTDL can be entered to the system via a, or the, user device 104 or another suitable input device, these test configuration files will be stored on the data storage device 108. Each test configuration file defines what augmented reality objects or virtual reality objects are used in a test, where they are located in the test environment and how they behave. Each test configuration file is typically, but not exclusively, associated with a test for a medical condition. The behaviour of the virtual reality objects being when and how the virtual objects are displayed, removed and how they interact with a real person, called collisions. For example, a virtual reality walkway may comprise a start line and end line and when the subject is standing on the start line it changes colour to indicate the correct position of the subject.

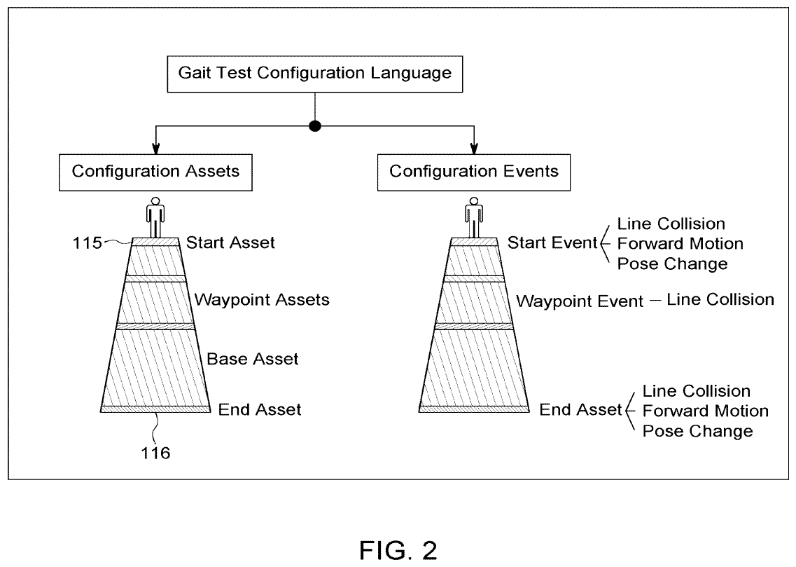

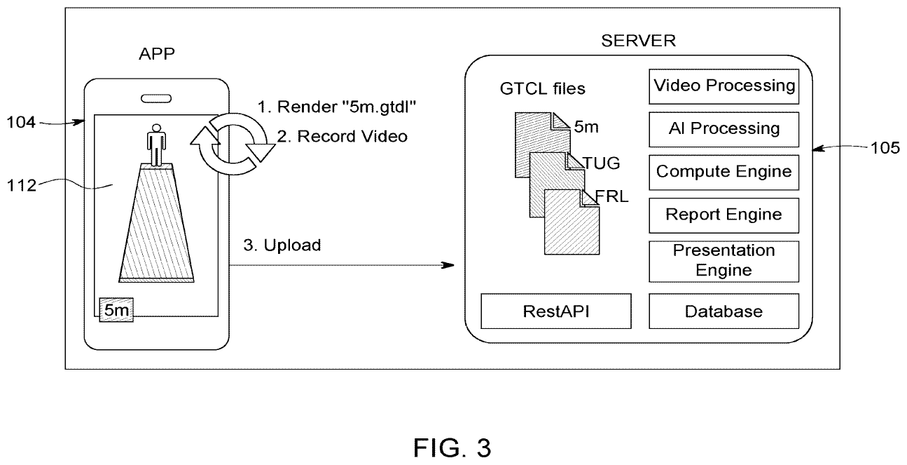

Referring now to FIGS. 1, 2 and 3, the user device 104 has an application 112, for example the GaitKeeper app, running which presents at user interface (UI) to a user. The application 112 may be downloaded from a dedicated server such as the processor 106 or from a commercially available app store. When launched the application 112 presents an app with a list of one or more possible gait tests that the user may want to perform. In use, a user selects a gait test from a menu of the application 112, the gait test loads the appropriate GTDL configuration file.

In one embodiment the application 112 comes with a number of preloaded GTDL configuration files stored locally on the user device 104 each of which corresponds to a gait analysis test. Alternatively, or additionally, the user device 104 may download the selected GTDL configuration file from the server 105 if the user device 104 does not have the desired configuration file stored upon it. Typically, the GTDL configuration files are generated using machine learning techniques using subjects without known medical conditions, providing a large pool of subjects and also maintaining ethical and privacy standards.

The application 112 causes the camera 102 to operate in video mode and displays the output of the camera 102 on a screen 114 of the user device 104. Once the GTDL configuration file is run on the user device 104 augmented reality objects such as a start line 115 and a finish line 116 are overlaid on the video feed displayed on the screen 114. It will be appreciated that other augmented reality objects, for example waymarkers, reference points or obstacles to be negotiated by the test subject. The subject then walks along a path defined by the augmented reality objects whilst the video feed is recorded on using the camera 102. A digital gait data file comprising the video feed and any associated metadata is forwarded to the server comprising the processor 106 and the data storage device 108, via the network 110 where it is stored on the data storage device 108. Typical, but non-exclusive, examples of meta-data can include any one or combination of video format descriptor (either mp4 or mov), video length descriptor (in seconds), accelerometer data feed from the camera, depth data feed from the camera.

The processor 106 of the server 105 runs a series of routines to process the gait data file using a declarative programming language, referred to hereinafter as “Gait Test Compute Language” (GTCL), GTCL is typically enacted in JSON and defines a range of motion and gait parameter computations that are computed by the system.

Table 2 shows an exemplary the GTCL language schema.

TABLE 2

| | |
|---|---|
| Test Name | The name of the test configuration. |
| | This is used to present the test name in the user interface. |
| | [String of four or less characters] |
| Test | A textual description of what this assessment does. |
| Description | It is used to show an information dialog in the user |
| | interface. |
| | [String: 0-200 characters] |
| OperationArray | A list of computations to carry out - represented using the |
| | operation syntax |

Operation Syntax
Operations are presented using the following syntax
{OperationName, Input Arguments, OutputLabel}

| | |
|---|---|
| 2PAngle | {2PAngle, {Point1, Point2}, outputLabel} |
| 3PAngle | {3PAngle, {Point1, Point2, Point3}, outputLabel} |
| 2PDist | {2PDist, {Point1, Point2}, outputLabel} |
| Convex | {CH/CD, {Point1 . . . Pointn}, |
| Hull/Centroid | outputLabel, StartFrame, EndFrame} |
| CO | {CO {Arithmetic operation using outputLabel}, |
| | OutputLabel} |
| PD | {PD, Start Frame, End Frame, {Point Array}, {Point |
| | Angle Array}, OutputLabel} |

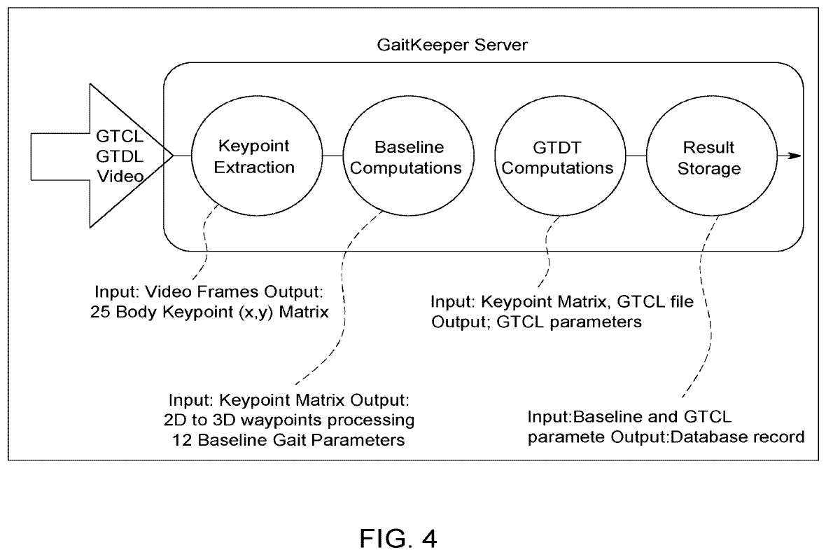

Referring now to FIG. 4, one routine is an artificial intelligence engine to extract keypoint data that mirrors, or approximates closely to, joints between bones of the subject, for example, by way of non-limiting example, knee joints, elbow joints, or other important points for gait analysis, for example, by way of non-limiting example, the head or centre of the torso. This keypoint analysis can be carried out using machine learning algorithms, such as, by way of non-limiting examples, convolution neural networks, optical flow or hough classifiers, to identify specified points of interest within a subject type, for example human, cow etc. The artificial intelligence engine also uses the augmented reality or virtual reality objects as reference points from which to calculate depths of the subject, i.e. how far from the start point the subject is at any given point in the test, thereby producing a spatio temporal multi-dimensional data structure from the video imagery. The video is captured with the augmented reality object visible, and embedded in the video data, and artificial intelligence detects the start line and end line and when the subject passes over them. The three dimensional nature of the data structure allows the keypoints to be calculated accurately such that their subsequent processing provides more accurate gait analysis.

A compute engine running on the server 105 uses GTCL to calculate a number of relationships between intra-frame characteristics of the subject and also inter-frame relationships between those characteristics. For example, within a single frame the compute engine can calculate, by way of non-limiting example, the angle between two points and a vertical or horizontal axis, the angle between three points, the distance between two points, the area of a convex hull of points, the centroid of a convex hull of points, the conjunctive joining of sub calculations, multi point pose detection, and between frames the compute engine can calculate, by way of non-limiting example, time between multi point Poses and acceleration of a point between frames (in conjunction with waypoint data). A new computation can be composed by sending the output of one computation to the input of the next. For example, a convex hull algorithm computes the shape of something (it returns a polygon), the polygon is passed to a centroid function to fix the centre of that shape, so called pipelining of calculations.

In use, the user records the subject using the camera 102 via the user device application, the start of the gait assessment by the processor commences when the subject crosses the virtual asset associated with the start line 115. Additionally, or alternatively, by way of non-limiting example, the forward motion of the subject between frames of the video recording or a pose change from sitting to standing is detected between frames of the video recording can be used to trigger commencement of the gait assessment. A waypoint event is triggered when the subject crosses an augmented reality waypoint in the video recording, as noted hereinbefore the waypoint can be used to calculate a virtual third dimension to the two dimensional pixelated image. The endpoint of the gait test is triggered when the subject crosses the virtual asset associated with the end line 116. Alternatively, or additionally, by way of non-limiting example the end point of the gait test can be triggered by a pose change from standing to sitting being detected. All events are represented as a triple (EventType, EventId, Time). Time is represented as number of video frames from the start of the video recording, multiplied by the video sampling rate.

Referring now further to FIG. 4, one embodiment of the processing of the gait data file following key point determination comprises making baseline computations: using the spatio temporal matrix of the keypoint and time calculations using the waypoint collection data to create three dimensional estimates for each video frame to provide intra-frame depth information. Using this data, for example, twelve baseline gait parameters are computed, non-limiting examples of gait parameters include gait Speed (average) (m/s), gait Speed (by gait cycle) (m/s), step count (number), step length (average) (m), step length (left side) (m), step length (right side) (m), feet support base (when feet in flat position) (m), heel strike events (time), toe off events (time), foot flat events (time), foot which moved forward first (time), time of forward movement (time). The spatio temporal matrix and depth information are subject to computations from the GTCL file to create a set of bespoke results. For example, the user can define a pipeline of operations to allow a set of measured parameters to feed into a user defined end result. The baseline and GTCL results are stored on the data storage device 106. The storing of these results allow for direct longitudinal comparison of objectively measured gait assessments in order that the progression of a condition of the subject can be tracked and monitored without subjective assessments.

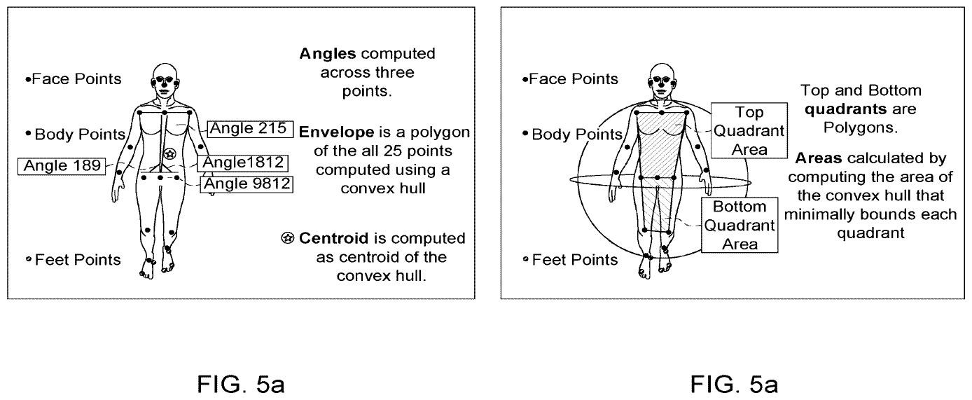

It is possible to select a subset of the key points for carrying out a gait assessment, this is defined in the GTCL file which is executed on the server 105. For example, as shown in FIGS. 5a subset of points can be selected or as shown in FIG. 5b a geometric figure can be defined and the motion of the subset and/or geometric figure can be used in the gait assessment. For example, 25 points on the body which are distributed across the head, arms, upper body, legs and feet may be selected for assessing a subject's gait. In some clinical evaluations, arm swing is measured to understand the symmetry of the movement. Arm swing is measured by computing the area of the three points that make up the arm and following the motion of that area in the captured video. In one, non-limiting, example, this is carried out for both the left arm and the right arm and then a comparison of left and right performed to determine the degree of symmetry in movement. Such a comparison of left to right bodily symmetry can be used in the fitting of a prosthetic fitting.

Table 3 shows a section of code that can be used to define such a subset of keypoints in GTCL.

```
TABLE 3
{“TestName”: “GKType1”,
“TestDesc”: “A stondord 4m walkway with Convex hull calculations”,
  “Operations”: [ {“1”,“CH”,{p7,p8,p9,p10,p11,p12},“GKType1-CH-Top”},
    {“2”,“CH”,{p10,p11,p12,p13,p14},“GKType1-CH-Bot”},
    {“3”,“CO”,{“CGKType1-CH-Top/GKType1-CH-Box)”},“GKType1-CH-Ratio”}
  ]
}
```

It will be appreciated that although described with reference to diagnostics the present invention can be used for security, for example to identify a person via their gait, or for sports coaching, for example, to improve an athlete's running style or improve their stability. It will be appreciated that the analytic technique can comprise a diagnostic test arranged to diagnose, by way of non-limiting example, a disease and/or a condition and/or a syndrome. Alternatively, or additionally, the analytic technique can be arranged to, by way of non-limiting example, analyse a user's gait in respect of sporting performance and/or determine the identity of a user by comparison to a library of known users' gaits.

In general, the routines executed to implement the embodiments of the invention, whether implemented as part of an operating system or a specific application, component, program, object, module or sequence of instructions, or even a subset thereof, may be referred to herein as “computer program code,” or simply “program code.” Program code typically comprises computer readable instructions that are resident at various times in various memory and storage devices in a computer and that, when read and executed by one or more processors in a computer, cause that computer to perform the operations necessary to execute operations and/or elements embodying the various aspects of the embodiments of the invention. The computer readable program instructions for carrying out operations of the embodiments of the invention may be, for example, assembly language or either source code or object code is written in any combination of one or more programming languages.

The program code embodied in any of the applications/modules described herein is capable of being individually or collectively distributed as a program product in a variety of different forms. In particular, the program code may be distributed using the computer readable storage medium having the computer readable program instructions thereon for causing a processor to carry out aspects of the embodiments of the invention.

Computer readable storage media, which is inherently non-transitory, may include volatile and non-volatile, and removable and non-removable tangible media implemented in any method or technology for storage of information, such as computer-readable instructions, data structures, program modules, or other data. Computer readable storage media may further include RAM, ROM, erasable programmable read-only memory (EPROM), electrically erasable programmable read-only memory (EEPROM), flash memory or other robust state memory technology, portable compact disc read-only memory (CD-ROM), or other optical storage, magnetic cassettes, magnetic tape, magnetic disk storage or other magnetic storage devices, or any other medium that can be used to store the desired information and which can be read by a computer. A computer-readable storage medium should not be construed as transitory signals per se (e.g., radio waves or other propagating electromagnetic waves, electromagnetic waves propagating through a transmission media such as a waveguide, or electrical signals transmitted through a wire). Computer readable program instructions may be downloaded to a computer, another type of programmable data processing apparatus, or another device from a computer readable storage medium or an external computer or external storage device via a network.

Computer readable program instructions stored in a computer readable medium may be used to direct a computer, other types of programmable data processing apparatus, or other devices to function in a particular manner, such that the instructions stored in the computer readable medium produce an article of manufacture including instructions that implement the functions/acts specified in the flowcharts, sequence diagrams, and/or block diagrams. The computer program instructions may be provided to one or more processors of a general-purpose computer, special purpose computer, or other programmable data processing apparatus to produce a machine, such that the instructions, which execute via the one or more processors, cause a series of computations to be performed to implement the functions and/or acts specified in the flowcharts, sequence diagrams, and/or block diagrams.

In certain alternative embodiments, the functions and/or acts specified in the flowcharts, sequence diagrams, and/or block diagrams may be re-ordered, processed serially, and/or processed concurrently without departing from the scope of the invention. Moreover, any of the flowcharts, sequence diagrams, and/or block diagrams may include more, or fewer blocks than those illustrated consistent with embodiments of the invention.

The terminology used herein is for the purpose of describing particular embodiments only and is not intended to be limiting of the embodiments of the invention. As used herein, the singular forms “a”, “an” and “the” are intended to include the plural forms as well, unless the context indicates otherwise. It will be further understood that the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “includes”, “having”, “has”, “with”, “comprised of”, or variants thereof are used in either the detailed description or the claims, such terms are intended to be inclusive in a manner similar to the term “comprising”.

While a description of various embodiments has illustrated all of the inventions and while these embodiments have been described in considerable detail, it is not the intention to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the spirit or scope of the general inventive concept.

1. A gait analysis system comprising:
a video camera and a processor, the video camera and the processor being in communication and wherein the video camera is arranged to capture one or more video images of a subject's gait, and the processor is arranged process a data structure corresponding to video frames and reference points to impart perspective to the one or more video images at the processor, the processor being configurable to define test parameters associated with a video capture for a plurality of gait assessments.

2. The gait analysis system of claim 1, wherein the processor is arranged to analyse the one or more video images to determine a condition of a subject.

3. The gait analysis system of claim 1, wherein the processor is configurable to generate an augmented or virtual representation of the test parameters upon a screen associated with the gait analysis system.

4. The gait analysis system of claim 1, wherein a user interface is arranged to receive user inputs to define augmented or virtual reality objects to be used in a gait analysis, and
wherein the user interface is further arranged to define placement of the augmented or virtual reality objects and/or their behaviour within the data structure comprising the one or more video images, and
wherein the user interface is arranged to allow selection of analysis of at least one portion of the subject.

5. (canceled)

6. The gait analysis system of claim 3, wherein the processor is arranged to use the augmented or virtual reality objects as the reference points from which to determine one or more key points location in three dimensions from the one or more video images.

7. (canceled)

8. The gait analysis system of claim 4, wherein the portion of the subject comprises a set of key points associated with one or more joints between bones of a subject.

9. (canceled)

10. The gait analysis system of claim 4, wherein the portion comprises a fraction of the subject.

11. The gait analysis system of claim 1, comprising a data storage device linked to the processor, the data storage device being arranged to store a plurality of data sets each of which corresponds to a pre-configured computational analytic techniques.

12. The gait analysis system of claim 11, wherein the processor is arranged to run one or more pre-configured computational analytic techniques on captured video images.

13. The gait analysis system of claim 11, wherein a user interface is arranged to allow selection of any one or combination of pre-configured computational analytic routines to be run by the processor upon the captured video.

14. The gait analysis system of claim 1, wherein the processor is arranged to perform gait analysis based solely on the one or more video images, wherein neither the subject nor a subject's environment comprises external physical markers denoting points to be monitored.

15. (canceled)

16. (canceled)

17. A computer implemented method for a gait analysis of a subject comprising:
i) defining a gait analysis test via a user interface of a user device;
ii) generating augmented or virtual reality reference points associated with a test path at a processor based on the gait analysis test defined in step (i);
iii) recording a sequence of video frames of the subject perambulating; and
iv) processing a data structure corresponding to the video frames and the reference points to impart perspective to one or more video images at the processor.

18. The method of claim 17, further comprising;
configuring the processor to define test parameters associated with video capture for a plurality of gait assessments associated with a plurality of conditions.

19. The method of claim 17, further comprising:
receiving user inputs to define augmented or virtual reality objects to be used in the gait analysis;
defining a placement of the augmented or virtual reality objects and/or their behaviour within the data structure;
displaying an augmented or virtual representation of test parameters upon a screen associated with a gait analysis system;
using the augmented or virtual reality objects as reference points from which to determine one or more key points location in three dimensions from the one or more video images; and
wherein the one or more key points are associated with one or more joints between bones of the subject.

20. (canceled)

21. (canceled)

22. (canceled)

23. (canceled)

24. The method of claim 17, further comprising:
selecting analysis of at least one portion of the subject comprising a set of key points.

25. (canceled)

26. The method of claim 17, further comprising:
storing a plurality of data sets each of which corresponds to an analytic technique at a data storage device associated with the processor.

27. (canceled)

28. The method of claim 26, further comprising:
executing one or more pre-configured computational analytic techniques on [the] captured video images.

29. The method of claim 17, further comprising:
performing the gait analysis based solely on the one or more video images, where neither the subject nor a subject's environment comprises external physical markers denoting points to be monitored.

30. (canceled)

31. The method of claim 17, further comprising:
analysing the video frames and/or the data structure to determine a condition of the subject based upon the gait analysis defined in step (i).

32. A non-transitory computer readable medium having instructions stored thereon, that when executed by a processor cause the processor to perform operations, the operations comprising:
i) defining a gait analysis test via a user interface of a user device;
ii) generating augmented or virtual reality reference points associated with a test path at the processor based on the gait analysis test defined in step (i);
iii) recording a sequence of video frames of a subject perambulating; and
iv) processing a data structure corresponding to the video frames and the reference points to impart perspective to video images at the processor.
